(19) United States
(12) Patent Application Publication
(10) Pub. No.: US 2020/0197635 A1
(43) Pub. Date: Jun. 25, 2020

(54) DEVICE WITH FLOW RATE INDICATOR

(71) Applicant: CLEMENT CLARKE INTERNATIONAL LIMITED, Harlow, Essex (GB)

(72) Inventors: David SPENCER, Harlow, Essex (GB); Ronald BRUIN, Harlow, Essex (GB); Mark SANDERS, Dunstable, Bedfordshire (GB)

(73) Assignee: CLEMENT CLARKE INTERNATIONAL LIMITED, Harlow, Essex (GB)

(21) Appl. No.: 16/640,087

(22) PCT Filed: Aug. 3, 2018
(86) PCT No.: PCT/EP2018/071149

§ 371 Date: Feb. 19, 2020

(30) Foreign Application Priority Data
Aug. 22, 2017 (GB) 1713477.6

(51) Int. Cl. A61M 15/00

(52) U.S. Cl. CPC A61M 15/009; A61M 15/0021; A61M 2205/581; A61M 2205/3334; A61M 15/0091

(57) ABSTRACT

A drug receptacle for use with a respiratory inhaler device. The drug receptacle has a fluid flow rate indicator on an outer surface thereof. The fluid flow rate indicator comprises a corrugated portion having at least one and preferably a plurality of corrugations extending radially from the outer surface of the drug receptacle. The flow rate indicator is operable to generate a sound signal. The present invention also relates to a corrugated flow rate indicator having a connection portion for connection to the outer surface of a drug receptacle.

### FIELD OF THE INVENTION

The present invention relates to a device for indicating a predetermined fluid flow rate. In particular, the present invention relates to a device for indicating a predetermined air flow rate during inhalation and/or exhalation. For example, the present invention relates to drug delivery inhaler devices, such as pressurised metered dose inhaler (pMDI) devices. The invention also relates to methods of operation of such devices.

### BACKGROUND OF THE INVENTION

It is desirable to provide an indication of a fluid (air) flow rate through a device such as a respiratory inhaler (e.g. a pressurised metered dose inhalers (pMDI)) to monitor and/or facilitate correct usage of the device.

GB-A-2372704 discloses a device for providing an indication of the respiratory flow rate of a patient. The device includes two reeds adapted to generate an audible signal at different air flow speeds through the device. The first reed generates an audible signal of a first pitch when the air flow reaches a predetermined minimum. The second reed generates an audible signal of a second pitch when the air flow reaches a predetermined maximum. Thus, the patient is informed when the air flow is within a desirable range, between the predetermined minimum and maximum.

Lavorini et al (2010) [F. Lavorini, M. L. Levy, C. Corrigan and G. Crompton, “The ADMIT series—issues in inhalation therapy. 6) Training tools for inhalation devices” Primary Care Respiratory Journal (2010) 19(4) 335-341] set out a review of training tools for inhalation devices, including the device disclosed in GB-A-2372704, referred to as the “2Tone” trainer.

Lavorini et al (2010) comment that two of the most critical patient errors in the uses of pMDI devices are a failure to coordinate inhalation with actuation of the device and inhaling the aerosolized drug too quickly. This is considered to be a critical issue—incorrect use of a pMDI device means that the drug delivered to the patient is being delivered sub-optimally. In turn, this means that the patient does not receive the correct dose of the drug, which can lead to serious problems in the ongoing treatment of conditions such as asthma.

GB-A-2490770 discloses a pMDI actuator body and a spacer for a pMDI inhaler that incorporates an air flow rate indicator comprising a reed which oscillates and generates a sound signal at a predetermined minimum level suitable for delivery of the drug to the patient.

There is a desire to provide an improved air flow rate indicator for such devices (e.g. a respiratory inhaler) that has a simple construction thus facilitating manufacture and reducing manufacturing costs.

There is also a desire for a system/method that monitors a patient's usage of such a device (e.g. a respiratory inhaler) and, in particular, records air flow rates at the point of actuation of the device and the duration of the optimal flow rate for drug delivery after actuation.

### SUMMARY OF THE INVENTION

In a first aspect, the present invention provides a drug receptacle for use with a respiratory inhaler device, the drug receptacle having a fluid flow rate indicator on an outer surface thereof.

Typically, the drug receptacle is a drug canister, e.g. a pMDI drug canister.

The inventors have found that when a drug receptacle having a fluid flow rate indicator on its outer surface is used within a respiratory inhaler device having a fluid flow path therethrough, the flow rate indicator can be used to provide an indication of when the fluid flow rate along the fluid flow path is at a predetermined minimum level suitable for delivery of the drug to the patient.

Optional features of the invention will now be set out. These are applicable singly or in any combination with any aspect of the invention.

A drug receptacle e.g. a drug canister for use with a pMDI respiratory inhaler typically has a tubular, e.g. cylindrical, body with one closed axial end and one open axial end, the open axial end for receiving a metering valve. Such a drug receptacle is configured to be received in an axial orifice in a respiratory inhaler device, the axial orifice partly defining the fluid flow path through the device. The fluid flow path within the axial orifice of the respiratory inhaler will typically be parallel to the axis of the tubular body of the drug receptacle. Typically, the fluid flow path passes along a gap formed between an outer surface of the tubular body, and an inner surface of the respiratory inhaler, e.g. along a gap formed between the tubular body and the axial orifice. Preferably, the gap extends over at least the flow rate indicator. In some embodiments, the gap may be an annular gap.

Typically, the fluid flow rate indicator is an air flow rate indicator.

In some embodiments, the fluid flow rate indicator comprises a corrugated portion having at least one and preferably a plurality of corrugations extending radially from the outer surface of the drug receptacle (e.g. radially from the outer surface of the tubular body), the flow rate indicator being operable to generate a sound signal.

The inventors have found that providing a fluid flow rate indicator comprising a corrugated portion having at least one and preferably a plurality of corrugations for extending radially into the fluid flow path induces turbulent flow in a fluid moving along the fluid flow path when the fluid flow rate is above the predetermined rate. The turbulent flow produced generates the sound signal which can provide an indication that the predetermined flow rate has been achieved.

Without wishing to be bound to any theory, the inventors believe that laminar flow of fluid (e.g. gas/air) occurs along the fluid flow path through the respiratory inhaler device at fluid flow rates below the predetermined fluid flow rate. As the fluid flow rate increases, the peak(s) and trough(s) of the corrugated portion induce turbulent eddies in the fluid until, at the predetermined fluid flow rate, sound oscillations are generated which match the resonant frequency of the corrugated portion of the fluid flow rate indicator and thus generate a sound signal (which may or may not be audible to the human ear). The sound signal has a narrow frequency and detection of this frequency sound signal (either by the human ear and/ or through software for audible sound signals, or through software for non-audible sound signals) can provide a clear indication of when the predetermined fluid flow rate has been achieved along the fluid flow path.

The corrugated portion may form at least part (or even the whole of) the outer wall surface of the tubular body portion of the drug receptacle. The corrugated portion may be integrally formed (e.g. moulded) as part of the outer surface of the tubular body portion of the receptacle. By providing a corrugated fluid flow rate indicator integrally formed with the receptacle, e.g. formed/moulded on the exterior surface, the device has a simple construction with minimal components and no moving parts.

The outer surface of the drug receptacle (e.g. the outer surface of the tubular body portion of thereceptacle) may be substantially smooth (uncorrugated) in areas other than in the corrugated portion.

Alternatively, the corrugated portion may be retrofitted to the drug receptacle (i.e. drug canister), e.g. retrofitted to an outer surface of the drug canister.

The flow rate indicator/corrugated portion may be separately formed and affixed to the outer surface of the drug receptacle. For example, the flow rate indicator may comprise a sleeve or clip into which the receptacle (e.g. the tubular body portion of a canister) is inserted or the flow rate indicator may comprise an adhesive portion for affixing to the outer surface of the drug receptacle.

In some embodiments, the corrugated portion may completely encircle the outer surface of the drug receptacle/tubular body of the drug receptacle. In other embodiments, the corrugated portion may only partially encircle the outer surface of the drug receptacle.

In some embodiments, the corrugated portion may extend the entire axial length of the drug receptacle e.g. the entire axial length of the tubular body of a drug canister. In other embodiments, the corrugated portion may extend only along a portion of the axial length of the drug receptacle.

In some embodiments, the corrugated portion may have an axial length (extending parallel to the axis of the tubular body/air flow path) of between 2 and 70 mm, such as between 2 and 60 mm, between 2 and 50 mm, between 2 and 40 mm, between 2 and 30 mm, between 2 and 20 mm, between 10 and 60 mm, between 10 and 50 mm, between 10 and 40 mm, between 10 and 30 mm, between 10 and 20 mm, between 20 and 60 mm, between 20 and 50 mm, between 20 and 40 mm, between 20 and 30 mm, between 30 and 60 mm, between 30 and 50 mm, between 30 and 40 mm, for example around 33-36 mm. The inventors have found that providing a corrugated portion having an axial length of at least 30 mm can provide an air flow rate indicator that generates two (or more) sound signals (of differing frequencies) within an air flow rate range associated with human desirable drug inhalation (e.g. in the range of 20-60 l/min). This can be used, for example, to indicate the range of air flow rates suitable for optimal drug delivery with the first sound signal being generated at the predetermined minimum level and a second sound signal being generated at a predetermined maximum level.

The corrugated portion may comprise a plurality of parallel ridges/peaks spaced by a plurality of troughs/furrows which at least partially encircle the drug receptacle (e.g. the tubular body of the drug receptacle).

The plurality of ridges/troughs may be oriented substantially perpendicularly to the axis of the tubular body of the drug receptacle (and therefore substantially perpendicularly to the fluid flow path) or they may be at an angle to the tubular body of the drug receptacle/fluid flow path.

In other embodiments, the corrugated portion comprises a spiral or screw-thread ridge/peak which encircles the tubular body of the drug receptacle.

In some embodiments, the corrugated portion comprises between 1 and 20 corrugations such as between 7 and 12 corrugations, e.g. between 7 and 10 corrugations, for example 8 corrugations (i.e. 8 peaks/ridges and associated troughs/furrows) or 9 corrugations (i.e. 9 peaks/ridges and associated troughs/furrows).

The pitch of the corrugations i.e. the spacing between adjacent peaks may be between 2-5 mm e.g. around 3 mm.

The height of the corrugations i.e. the height from the base of a trough to the apex of the peak may be between 0.5 and 2.0 mm, for example between 0.5 and 1.0 mm e.g. around 0.6 mm.

In some embodiments, the or each ridge in the corrugated portion has an unsymmetrical longitudinal cross-sectional profile (i.e. the cross-sectional profile parallel to the direction of fluid flow). For example, the or each ridge may have a substantially sawtooth/shark fin profile with differing gradients on opposing (upstream/downstream) sides. The apex of the or each ridge is preferably rounded.

By providing an asymmetrical ridge, the flow rate indicator can be used to produce a first sound signal when fluid flows in a first direction and a second sound signal when fluid flows in a second direction. The first and second sound signals could have different frequencies.

In some embodiments, the corrugated portion extends to the closed axial end of the tubular body of the drug receptacle.

In other embodiments, the corrugated portion is spaced from the closed axial end of the tubular body of the drug receptacle.

In preferred embodiments, the corrugated portion comprises a lead-in portion at its axial end, the lead-in portion comprising the or one of the ridges such that as fluid first enters the corrugated portion it enters on a “rising-slope” and is directed away from the axis of the tubular body by the inclined surface of the or one of the ridges.

Some embodiments comprise a plurality of corrugated portions as described above. The corrugated portions may be axially spaced along the tubular body of the drug receptacle with un-corrugated e.g. smooth outer surface of the tubular body of the receptacle interposed between the corrugated portions. Alternatively, they may be circumferentially spaced around the tubular body of the receptacle.

In a second aspect, the present invention provides a fluid flow rate indicator having a connection portion for connection to an outer surface of a drug receptacle for use with a respiratory inhaler device.

Typically, the connection portion is for connection to an outer surface of a drug canister, e.g. a pMDI drug canister. The flow rate indicator may be configured to be retrofitted to drug canister, e.g. an ‘off-the-shelf’ drug canister.

As described above, a drug receptacle e.g. a drug canister for use with a pMDI respiratory inhaler typically has a tubular, e.g. cylindrical, body with one closed axial end and one open axial end, the open axial end for receiving a metering valve.

In some embodiments, the connection portion forms or is formable into a complete or partial sleeve defining a bore for housing at least part of the tubular body of the drug receptacle. The connection portion may be rigid. Alternatively, the connection portion may be flexible.

The sleeve may be a full sleeve (i.e. for completely encircling the receptacle) or a partial sleeve (i.e. for only partially encircling the receptacle).

For example, the connection portion may be a sleeve or clip defining a bore for forming an interference fit with the tubular body of the drug receptacle. The sleeve or clip may be rigid.

In other embodiments, the connection portion may be flexible for flexing into a profile conforming to at least a portion of the outer surface of the drug receptacle (thus defining a bore for housing at least part of the tubular body of the drug receptacle). In these embodiments, the connection portion comprises an adhesive layer for adhesively securing the connection portion to the outer surface of the drug receptacle.

The flow rate indicator may comprise a corrugated portion provided on an outer surface of the connection portion (i.e. on the surface distal the bore for housing the drug receptacle), the corrugated portion having at least one and preferably a plurality of corrugations.

The corrugated portion of the flow rate indicator of the second aspect may be as described for the first aspect. A drug receptacle having the fluid flow rate indicator of the second aspect applied to it may function substantially as described for the first aspect.

For example, the corrugated portion may have at least one and preferably a plurality of corrugations extending radially away from the connection portion, the flow rate indicator being operable to generate a sound signal.

The corrugated portion may completely encircle the outer surface of the connection portion (e.g. so as to completely encircle the drug receptacle/tubular body of the drug receptacle in use). In other embodiments, the corrugated portion may only partially encircle the outer surface of the connection element (and therefore the drug receptaclewhen in use).

In some embodiments, the corrugated portion may extend the entire axial length of the bore formed or formable by the connection portion. In other embodiments, the corrugated portion may extend only along a portion of the axial length.

In some embodiments, the corrugated portion may have an axial length of between 2 and 70 mm, between 2 and 60 mm, between 2 and 50 mm, between 2 and 40 mm, between 2 and 30 mm, between 2 and 20 mm, between 10 and 60 mm, between 10 and 50 mm, between 10 and 40 mm, between 10 and 30 mm, between 10 and 20 mm, between 20 and 60 mm, between 20 and 50 mm, between 20 and 40 mm, between 20 and 30 mm, between 30 and 60 mm, between 30 and 50 mm, between 30 and 40 mm, for example around 33-36 mm.

The corrugated portion may comprise a plurality of parallel ridges/peaks spaced by a plurality of troughs/furrows which at least partially encircle the connection portion.

The plurality of ridges/troughs may be oriented substantially perpendicularly to the axis of the bore formed or formable by the connection element or they may be at an angle to the axis or the bore.

In other embodiments, the corrugated portion comprises a spiral or screw-thread ridge/peak which encircles the connection element sleeve.

In some embodiments, the corrugated portion comprises between 1 and 20 corrugations such as between 7 and 12 corrugations, e.g. between 7 and 10 corrugations, for example 8 corrugations (i.e. 8 peaks/ridges and associated troughs/furrows) or 9 corrugations (i.e. 9 peaks/ridges and associated troughs/furrows).

The pitch of the corrugations i.e. the spacing between adjacent peaks may be between 2-5 mm e.g. around 3 mm.

The height of the corrugations i.e. the height from the base of a trough to the apex of the peak may be between 0.5 and 2.0 mm, for example between 0.5 and 1.0 mm e.g. around 0.6 mm.

In some embodiments, the or each ridge in the corrugated portion has an unsymmetrical longitudinal cross-sectional profile (i.e. the cross-sectional profile parallel to the direction of fluid flow). For example, the or each ridge may have a substantially sawtooth/shark fin profile with differing gradients on opposing (upstream/downstream) sides. The apex of the or each ridge is preferably rounded.

In preferred embodiments, the corrugated portion comprises a lead-in portion at its axial ends the lead-in portion comprising the or one of the ridges such that as fluid first enters the corrugated portion it enters on a “rising-slope” and is directed away from the axis of the tubular body by the inclined surface of the or one of the ridges.

Some embodiments comprise a plurality of corrugated portions as described above. The corrugated portions may be axially spaced along the outer surface of the connection portion with un-corrugated surfaces interposed between the corrugated portions. Alternatively, they may be circumferentially spaced around connection portion.

In a third aspect, the present invention provides a respiratory inhaler device for delivery of a drug to a patient, the device comprising:

an aperture for inlet of air into the device;
a mouthpiece for communication with the mouth of a patient;
a device body defining an air flow path extending from the aperture to the mouthpiece along which air is drawn to the mouthpiece by inhalation by the patient, the device body housing either a drug receptacle according to the first aspect or a drug receptacle fitted with a flow rate indictor device according to the second aspect.

Typically, the drug receptacle is a drug canister, e.g. a pMDI drug canister.

The air (fluid) flow path may extend along a gap formed between an outer surface of a tubular body portion of the drug receptacle, and an inner surface of the respiratory inhaler device, e.g. along a gap formed between a tubular body of the drug receptacle, and the respiratory inhaler device body. The gap may extend over at least the flow rate indicator. In some embodiments, the gap may be an annular gap.

In some embodiments, the inhaler device may be a pressurised metered dose inhaler (pMDI) device. In such devices, the drug (or combination of drugs) is typically provided in the form of a liquid in solution or suspension held in a (pressurised) drug canister. Actuation of the drug canister is typically achieved by depressing the canister downwards into the device body. This causes an interaction between the drug canister and a seat within the device body that causes a metered dose of liquid to be ejected from the drug canister, along with a propellant gas. In this manner, the liquid is aerosolized for inhalation by the patient.

Pressurised metered dose inhaler (pMDI) devices typically have a device body comprising an upright portion defining the axial orifice and extending from an aperture to a transverse mouthpiece for communication with the mouth of the patient. As well as allowing the inlet of air into the respiratory device, the aperture is adapted to receive the drug canister which is housed in the axial orifice of the upright portion thus occluding the air flow path. The seat for the location of the drug canister is typically provided at the junction between the upright portion and the transverse mouthpiece.

The upright portion is typically tubular (e.g. cylindrical). It may have a circular cross section. In some embodiments, it may have an oval or barrel-shaped cross section. It may have an internal diameter of 24-28 mm (but will be occluded by the drug canister such that the air flow path is restricted).

In some embodiments, the resistance of the axial orifice is between 0.3 and 3.6 kPa at a flow rate of 30 L/min and between 1.7 and 18.5 kPa at a flow rate of 60 L/min.

In preferred embodiments, the tubular upright portion is substantially cylindrical and dimensioned such that the drug canister forms a snug fit against the inner wall of the upright portion with the corrugated portion of the drug canister provided within the axial orifice (extending parallel to the air flow path) provided in the upright portion. This ensures that the air drawn along the air flow path by inhalation passes over the corrugated portion.

To use the pMDI, the patient will insert the mouthpiece into their mouth and inhale. The air flowing into the upright portion of the device body through the aperture will flow over the corrugated portion on the outer surface of the drug canister and into the transverse mouthpiece towards the patient's mouth. At the predetermined minimum flow rate, the air drawn along the air flow path will become turbulent as a result of the air tumbling over the peaks and troughs of the corrugated portion. When the oscillations match the resonant frequency of the corrugated portion, a sound signal having a narrow frequency width will be generated and the patient will know that the optimal inhalation rate has been achieved. The patient will then know to actuate/depress the drug canister to release the drug into the air flow path for inhalation.

The generation of the sound signal may be detected by ear by the patient or the patient may be provided with software (e.g. in the form of a mobile phone app) to detect the generation of the sound signal and thus the attainment of the predetermined minimum flow rate for optimal drug delivery.

Upon depression of the canister, the frequency/pitch of the sound signal may change as a result of the change in the resistance along the air flow path if the axial length of the corrugated portion opposed to the device body changes (e.g. increases). In situations where there is a desire to monitor patient compliance and/or monitor the alteration in the frequency/pitch of the sound signal, the sound signal could be monitored/recorded (e.g. by the computer software/mobile app) to detect the point of actuation of the canister. This would provide a cheap and easy-to-use method for monitoring patient usage which could capture not only the number of actuations but also record flow rates associated with actuations and the duration of the optimal air flow rate after actuation.

In a fourth aspect, the present invention provides a system comprising a device according to the third aspect and a sound receiver for detecting the sound signal.

In some embodiments, the sound receiver comprises computer software e.g. an application for running on a mobile device such as a smartphone app. The FrequenSee™ app, available as an Apple® and Android® app, may be used for detecting the sound signal.

In a fifth aspect, the present invention provides a method of monitoring actuation of a respiratory inhaler device for delivery of a drug to a patient, the method comprising:
providing a system according to the fourth aspect,
detecting the sound signal generated when the air flow rate along the air flow path is at or above the predetermined minimum level suitable for delivery of the drug to the patient,
detecting a change in frequency of the sound signal upon actuation of the device by the patient.

In some embodiments, the method comprises recording (e.g. using computer software such as an application for running on a mobile device such as a smartphone app) the duration of the change in the sound signal upon actuation by detecting the return to the original sound signal after actuation is complete.

In some embodiments, the method comprises recording (e.g. using computer software such as an application for running on a mobile device such as a smartphone app) the duration of the sound signal (e.g. the duration after actuation) to establish to duration of optimal inhalation by the patient.

This information can be used to monitor use of the inhaler by the patient. It can be used (either by the patient or by a healthcare provider) to ensure that actuation is being correctly coordinated with the optimal air flow rate through the device and that the optimal air flow rate is being maintained for a sufficient period of time after actuation. Current monitoring methods typically only monitor the number of actuations of the inhaler device and do not provide any information about the air flow rate at the time of actuation nor about the correct inhalation technique after actuation.

### BRIEF DESCRIPTION OF THE DRAWINGS

Embodiments of the invention will now be described by way of example with reference to the accompanying drawings in which:

FIG. 1 shows a longitudinal cross-sectional view through a first embodiment of a respiratory inhaler device according to an aspect of the present invention;

FIG. 2 shows a perspective view of a first embodiment of a flow rate indicator according to an aspect of the present invention;

FIG. 3a shows a perspective view of a second embodiment of a flow rate indicator according to an aspect of the present invention;

FIG. 3b shows the flow rate indicator of FIG. 3a, as applied to a drug canister

FIG. 4 shows a perspective view of a third embodiment of a flow rate indicator according to an aspect of the present invention;

FIG. 5a shows a perspective view of a fourth embodiment of a flow rate indicator according to an aspect of the present invention;

FIG. 5b shows the flow rate indicator of FIG. 5a, as applied to a drug canister; and

FIG. 6 shows a perspective view of a fifth embodiment of a flow rate indicator according to an aspect of the present invention.

### DETAILED DESCRIPTION AND FURTHER OPTIONAL FEATURES OF THE INVENTION

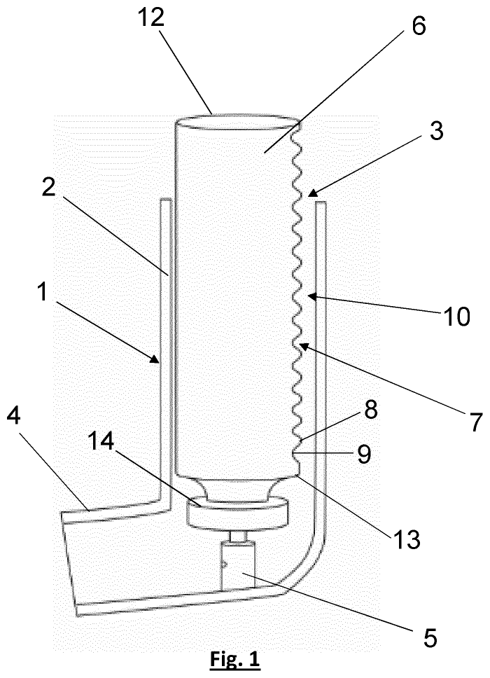

FIG. 1 shows a longitudinal cross-sectional view through a first embodiment of the present invention which comprises a pressurised metered dose inhaler (pMDI) 1 adapted to deliver respiratory drugs to a patient. The body of the pMDI 1 comprises an upright portion 2 having an aperture 3 for inlet of air into the pMDI and a transverse mouthpiece 4 for communication with the mouth of a patient. The upright portion 2 defines an air flow path extending from the aperture 3 to the transverse mouthpiece 4. The upright portion 2 is substantially cylindrical (with a substantially circular transverse cross-section) and the transverse mouthpiece 4 has a substantially oval or barrel-shaped transverse cross-section. This provides an oval or barrel-shaped mouthpiece 4 that can easily form a seal with the patient's mouth.

The upright portion has an internal diameter of around 24-28 mm.

The pMDI further comprises a seat 5 for location of a drug canister 6 containing a respiratory drug at the junction between the upright portion 2 and the transverse mouthpiece 4. The canister 6 is inserted into the upright portion 2 of the body through the aperture 3 and is housed in the upright portion 2.

The drug canister 6 comprises a tubular body having an integrally formed corrugated portion 7 which comprises a series of parallel ridges 8 and troughs 9. The ridges 8 and troughs 9 are integrally formed (moulded) into the outer surface of the tubular body of the drug canister 6 and are oriented substantially perpendicularly to the axis of the tubular body of the drug canister 6 and the air flow path 10.

The ridges 8 and troughs 9 partially encircle the canister 6 and extend the entire axial length of the tubular body of the drug canister from the closed axial end 12 to the open axial end 13 fitted with a metering valve 14 housed in the seat 5.

The axial length of the corrugated portion 7 is approximately 30 mm in length and comprises ridges 8 and troughs 9 having a pitch of 3 mm.

The corrugated portion 7 comprises a lead-in ridge (not pictured) at its axial end proximal the closed axial end 12 such that as air first enters the corrugated portion 7 it is directed away from the axis of the tubular body of the drug canister 6 by the inclined surface of the lead-in ridge.

To use the pMDI 1, the patient will insert the mouthpiece 4 into their mouth and inhale. The air flowing into the upright portion 2 of the body through the aperture 3 will flow around the canister 6, over the corrugated portion 7 and into the transverse mouthpiece.

At the predetermined minimum flow rate which is the minimum air flow rate for optimal drug inhalation, the air drawn along the air flow path will become turbulent as a result of the air tumbling over the ridges 8 and troughs 9 of the corrugated portion 7. When the oscillations match the resonant frequency of the corrugated portion of the body, a sound signal having a narrow frequency width will be generated and the patient will know that the optimal inhalation rate has been achieved.

The generation of the sound signal may be detected by ear by the patient or the patient may be provided with software (e.g. in the form of a mobile phone app) to detect the generation of the sound signal and thus the attainment of the predetermined minimum flow rate for optimal drug delivery.

When the optimal inhalation rate has been achieved, the patient will then know to actuate the drug canister 6 to release the drug into the air flow path for inhalation. Actuation of the canister 6 is typically achieved by depressing the canister 6 into the upright portion 2 of the body. This causes an interaction between the canister 6 and the seat 5 that causes a metered dose of liquid to be ejected from the canister 6, along with a propellant gas. The liquid is aerosolized, for inhalation by the patient. A drug of particular interest is salbutamol, marketed under the example trade names Ventolin™, Aerolin™, Ventorlin™, Asthalin™, Asthavent™, Proventil™ and ProAir™, for the management of asthma and other respiratory diseases.

Upon depression of the canister 6, the frequency/pitch of the sound signal will change as a result of the change in the axial length/geometry of the corrugated portion 7. In situations where there is a desire to monitor patient compliance and/or monitor the alteration in the frequency/pitch of the sound signal, the sound signal could be monitored/recorded (e.g. by the computer software/mobile app) to detect the point of actuation of the canister. The duration of the sound signal after actuation could also be monitored/recorded to help ensure that the optimal flow rate is maintained for a sufficient period of time after actuation.

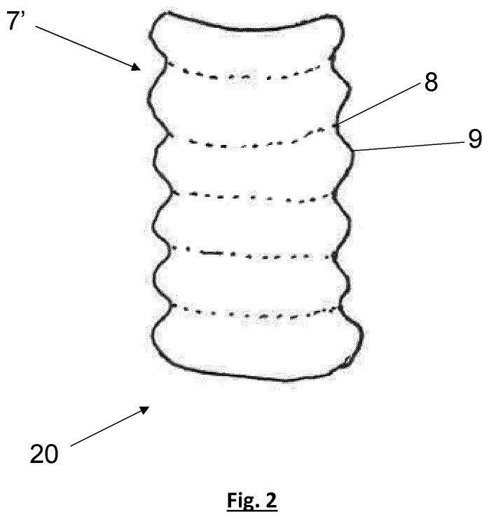

FIGS. 2 to 6 show flow rate indicators for fitting to an ‘off-the-shelf’ drug canister 6′.

FIG. 2 shows a flow rate indicator 20 having a flexible corrugated portion 7′ provided on a flexible adhesive connection portion (not shown). The connection portion/corrugated portion is flexible and can be deformed from a planar profile to a sleeve profile matching the profile of the outer surface of the drug canister and defining a bore for surrounding the outer surface of a drug canister. The adhesive layer can be used to affix the flow rate indicator to a drug canister. Flow rate indicator 20 may therefore be considered as being part of a label (e.g. adhesive label) for application to a drug canister.

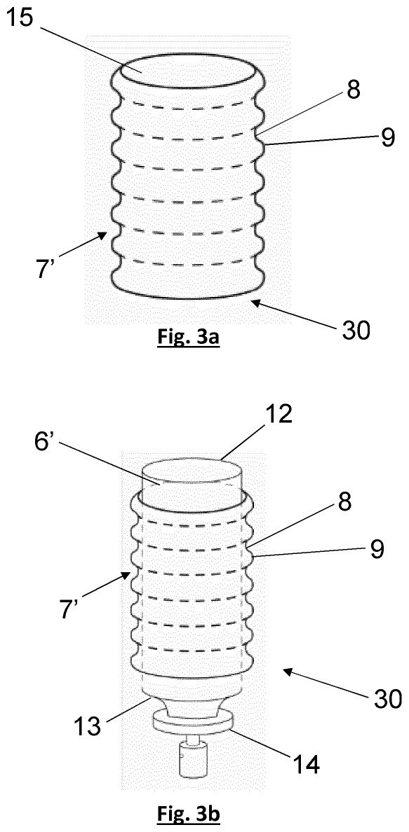

FIG. 3a shows a flow rate indicator 30 where the connection portion comprises a full, rigid sleeve defining a bore 15 into which a drug canister can be housed in an interference fit. Flow rate indicator 30 may be considered as being part of a sleeve (e.g. rigid sleeve) for application to a drug canister 6′.

FIG. 3b shows the flow rate indicator 30 of FIG. 3a, as applied to a drug canister 6′.

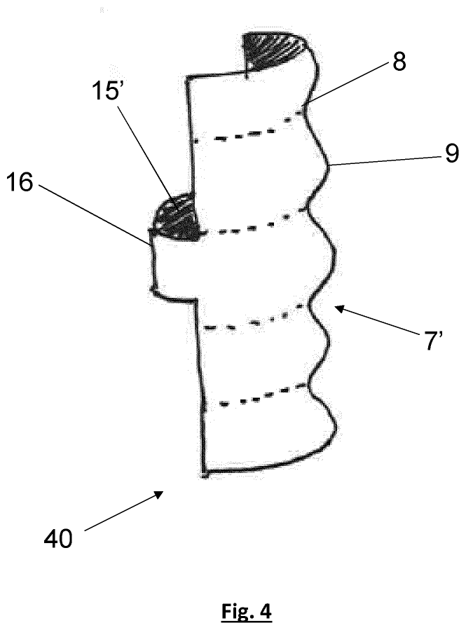

FIG. 4 shows a flow rate indicator 40 where the connection portion comprises a continuous clip portion 16 defining a bore 15′ into which a drug canister can be fitted. Flow rate indicator 40 may therefore be considered as being part of a clip (e.g. semi-rigid or rigid clip) for application to a drug canister.

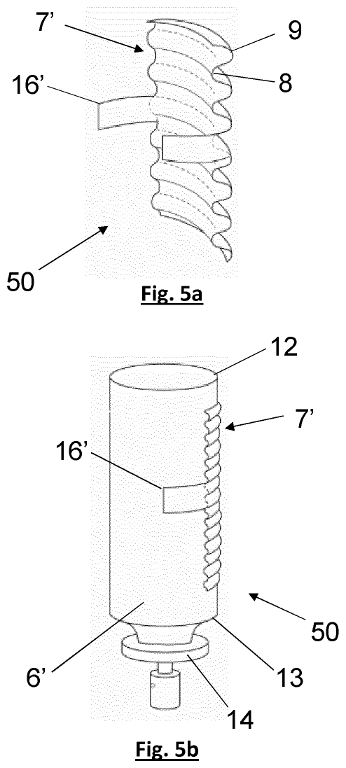

FIG. 5a shows a flow rate indicator 50, similar to the flow rate indicator 40 of FIG. 4, except for the fact that the clip portion 16′ comprises two arms, which jointly only extend part of the way around the body of the drug canister 6′, e.g. 50-60% of the way around. The arms of the clip portion 16′ are, as with the clip portion 16 of flow rate indicator 40, rigid or semi-rigid.

FIG. 5b shows the flow rate indicator of FIG. 5a, as applied to a drug canister 6′.

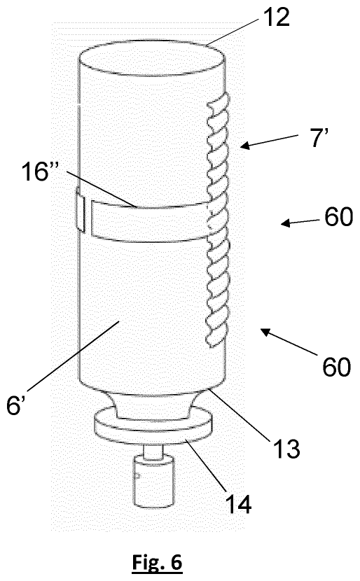

FIG. 6 shows a flow rate indicator 60, similar to the flow rate indicators 50 of FIG. 5, except for the fact that the two arms of the clip portion 16″ extend almost all of the way around the drug canister 6′, e.g. between 90-100% of the way around the drug canister.

pMDI drug canisters typically have a tubular body with a diameter of between 20 and 25 mm, and an axial length of between 30 and 100 mm.

The flow rate indicator may thus have a height, in the axial direction (e.g. the axial direction when mounted on a drug canister), of 10-100 mm. The flow rate indicator may have a width (e.g. in the circumferential direction when mounted on a drug canister) of up to 78 mm. Accordingly, the flow rate indicator may cover only a part of the tubular body portion of a drug canister (e.g. where the canister has a tubular length of 100 mm and a tubular diameter of 25 mm, and the flow rate indicator has a length of less than 100 mm, and/or a width of less than 78 mm).

Alternatively, the flow rate indicator can cover the entire tubular body portion of a drug canister (e.g. where the canister has a tubular length of 100 mm and a tubular diameter of 25 mm, and the flow rate indicator has a length of 100 mm, and/or a width of 78 mm).

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

All references referred to above are hereby incorporated by reference.

1. A drug receptacle for use with a respiratory inhaler device, the drug receptacle having a fluid flow rate indicator on an outer surface thereof.

2. A drug receptacle according to claim 1 wherein the fluid flow rate indicator comprises a corrugated portion having at least one and preferably a plurality of corrugations extending radially from the outer surface of the drug receptacle, the flow rate indicator being operable to generate a sound signal.

3. A drug receptacle according to claim 1 wherein the corrugated portion is integrally formed as part of the outer surface of the drug receptacle.

4. A drug receptacle according to claim 1 wherein the flow rate indicator/corrugated portion is separately formed and affixed to the outer surface of the drug receptacle.

5. A drug receptacle according to claim 1 wherein the corrugated portion completely encircles the outer surface of the drug receptacle.

6. A drug receptacle according to claim 1 wherein the corrugated portion extends the entire axial length of the drug receptacle.

7. A drug receptacle according to claim 1 wherein the corrugated portion has an axial length of between 2 and 70 mm.

8. A flow rate indicator having a connection portion for connection to an outer surface of a drug receptacle for use with a respiratory inhaler device.

9. A flow rate indicator according to claim 8 wherein the connection portion forms or is formable into a complete or partial sleeve defining a bore for housing at least part of the drug receptacle.

10. A flow rate indicator according to claim 8 wherein the connection portion is a sleeve or clip defining a bore for forming an interference fit with the tubular body of the drug receptacle.

11. A flow rate indictor according to claim 8 wherein the connection portion is flexible for flexing into a profile conforming to at least a portion of the outer surface of the drug receptacle.

12. A flow rate indicator according to claim 11 wherein the connection portion comprises an adhesive layer for adhesively securing the connection portion to the outer surface of the drug receptacle.

13. A flow rate indicator according to claim 8 wherein the flow rate indicator comprises a corrugated portion having at least one and preferably a plurality of corrugations provided on an outer surface of the connection portion.

14. A respiratory inhaler device for delivery of a drug to a patient, the device comprising:
an aperture for inlet of air into the device;
a mouthpiece for communication with the mouth of a patient;
a device body defining an air flow path extending from the aperture to the mouthpiece along which air is drawn to the mouthpiece by inhalation by the patient, the device body housing a drug receptacle according to claim 1.

15. A system comprising a device according to claim 14 and a sound receiver for detecting the sound signal.

16. A system according to claim 15 wherein the sound receiver comprises a computer software application for running on a mobile device.

17. A method of monitoring actuation of a respiratory inhaler device for delivery of a drug to a patient, the method comprising:
providing a system according to claim 15,
detecting the sound signal generated when the air flow rate along the air flow path is at or above the predetermined minimum level suitable for delivery of the drug to the patient,
detecting a change in frequency of the sound signal upon actuation of the device by the patient.

18. A method according to claim 17 further comprising recording the change in the sound signal upon actuation of the device.

19. A method according to claim 17 further comprising recording the duration of the sound signal after actuation of the device.

20. A respiratory inhaler device for delivery of a drug to a patient, the device comprising:
an aperture for inlet of air into the device;
a mouthpiece for communication with the mouth of a patient;
a device body defining an air flow path extending from the aperture to the mouthpiece along which air is drawn to the mouthpiece by inhalation by the patient, the device body housing a drug receptacle fitted with a flow rate indictor device according to claim 8.
